(19) United States
(12) Patent Application Publication
(10) Pub. No.: US 2002/0058921 A1
(43) Pub. Date: May 16, 2002

(54) Labial pad

(75) Inventor: Wayne Clement Sigl, Neenah, WI (US)

Correspondence Address: WILHELM LAW SERVICE, S.C., 100 W LAWRENCE ST, THIRD FLOOR, APPLETON, WI, 54911

(73) Assignee: Kimberly-Clark Worldwide, Inc., Neenah, WI

(21) Appl. No.: 10/037,377

(22) Filed: Dec. 31, 2001

Related U.S. Application Data
(63) Continuation-in-part of application No. 09/504,071, filed on Feb. 14, 2000.
Application No. 09/504,071 is a continuation-in-part of application No. 08/768,162, filed on Dec. 13, 1996, now abandoned.

(51) Int. Cl. A61F 13/15; A61F 13/20

(52) U.S. Cl. 604/385.201; 604/385.03; 604/387

(57) ABSTRACT

Absorbent articles and methods for use. The articles are used in the vestibule of a female wearer, and comprise a baffle, an absorbent, a retainer flap extending over the baffle, and optionally a liquid permeable cover. The absorbent article and retainer flap have first and second perimeters. A first portion of the second outer perimeter is positioned and attached proximate the first outer perimeter. The second portion of the second outer perimeter extends across an outer surface of the baffle. The first and second portions of the second perimeter can be sectioned. For disposal, the absorbent article is folded with first and second portions of the edge of the absorbent article in facing relationship. The retainer flap is folded over the edge portions to open the first cavity and define a second cavity receiving the facing portions.

This application is a continuation-in-part of application Ser. No. 09/504,071 filed Feb. 14, 2000, which is a continuation-in-part of application Ser. No. 08/768,162 filed Dec. 13, 1996, both of which are incorporated herein by reference in their entireties.

### BACKGROUND

The present invention relates generally to absorbent articles such as labial pads configured for disposition within the vestibule of a female wearer. More particularly, the present invention relates to labial pads having novel structure and methods for removing such pads from the vestibule of a wearer and/or novel structure and methods for preparing such labial pads for disposal.

A broad range and wide variety of absorbent articles configured for absorption of bodily exudates such as menstrual fluid are well known. With respect to feminine hygiene, the art has offered two basic types of feminine hygiene protection: namely sanitary napkins, developed for external wear about the pudendal region, and tampons, developed for residence within the vaginal cavity, and accordingly for interruption of menstrual flow therefrom prior to such menstrual flow reaching the vestibule. Hybrid feminine hygiene protection devices, attempting to merge the structural features of both sanitary napkins and tampons in a single type of device, have also been proposed, but have not seen a meaningful measure of acceptance insofar as the effort to achieve advantages with such devices has been overshadowed by the more demonstrable perpetuation of structural and anatomically functional disadvantages. Other less intrusive devices, known as labial or interlabial devices or pads and characterized by residing primarily within the wearer's vestibule while having a portion which at least partially resides external of the wearer's vestibule, have also been proposed.

Many of these prior devices have not fully satisfied the demand of consumers for smaller devices which may be worn interlabially by female wearers. In response thereto, several manufacturers have produced labial devices or pads which are quite small in size in comparison to the prior devices described above. However, the construction of many of these devices appears to fail to provide for a desired level of cleanliness of the wearer's hands during positioning of such device for wearing use, and for removal and disposal of such labial pads.

A significant factor affecting consumer acceptance is the ease of use, including ease and cleanliness attending proper placement of the labial pad in the vestibule of the wearer of such pad, and/or removal of the labial pad from the wearer's body, and disposal of the pad, after use. Typically, the wearer grasps the labial pad with her fingers and disposes it into proper placement within her vestibule. The wearer may also need to grasp the labial pad for removal, particularly if the labial pad is not expelled during urination. For placement of the pad in the vestibule, and/or for removal of such pad from the vestibule, for hygiene reasons, it is desirable that the wearer not touch the body-facing surface of the labial pad.

Disposal of a used labial pad presents its own unique challenges. Typically, the wearer grasps the labial pad in the vestibule for removal. However, in grasping a conventional labial pad in the vestibule, the user typically places one finger on the body-facing side of the pad as, typically, the side of the labial pad opposing the body-facing side carries no grasping structure. However, the body-facing side of the used labial pad bears body exudate fluids which are preferably not touched, for hygiene reasons.

Therefore, there is a need for improved labial pads, and for corresponding methods which provide for easily and hygienically removing such labial pad from a wearer's body without soiling the wearer or her clothing.

Further, there is a need for a labial pad which includes an attached retainer flap which can be used to hold and manipulate such pad, to receive and retain at least folded over portions of the labial pad, and to generally obstruct flow of body exudates from a used such labial pad.

Thus, a need exists for an improved labial pad which facilitates a desired level of cleanliness and hygiene of the wearer's hands during placement of a labial pad into an appropriate position within the vestibule as well as during removal of such labial pad from the vestibule and disposal of such labial pad.

### SUMMARY

In general, the invention relates to a family of novel absorbent articles. Such absorbent articles comprise a liquid impermeable baffle, an absorbent, a retainer flap extending over the baffle such that the baffle is between the retainer flap and the absorbent, and optionally a liquid permeable cover, such that the absorbent is between the cover and the baffle . The absorbent article is configured for disposition primarily within the vestibule of a female wearer. The absorbent article has a central longitudinal axis, an outer edge defining a first outer perimeter of the absorbent article, a body-facing surface, a surface opposed to the body-facing surface, a length, and a width. The retainer flap has a second outer perimeter. The second outer perimeter has first and second portions. The first portion is disposed proximate the first outer perimeter of the absorbent article, and is generally attached in the absorbent article at or adjacent the first outer perimeter. The second portion of the outer perimeter of the retainer flap extends across an outer surface of the baffle whereby the retainer flap covers a portion of the baffle. The second portion of the second outer perimeter of the retainer flap is displaceable from the baffle thereby to define a first cavity between the retainer flap and the baffle.

In preferred embodiments, the retainer flap is disposed generally on a first side of the central longitudinal axis, and extends along the length of the absorbent article.

In some embodiments, the second portion of the second outer perimeter of the retainer flap extends generally parallel to the central longitudinal axis.

In other embodiments, the second portion of the second outer perimeter of the retainer flap extends in a direction not parallel to the central longitudinal axis.

In some embodiments, the retainer flap forms a portion of the surface opposed to the body-facing surface.

In some embodiments the retainer flap comprises at least about 20 percent, preferably at least about 40 percent, of the surface opposed to the body-facing surface of the absorbent article.

In preferred embodiments, for disposal, the absorbent article is adapted to be folded upon itself such that first and second opposing portions of the outer edge at the first outer perimeter are brought into face-to-face relationship with each other, and wherein the retainer flap is adapted to be folded over the opposing portions of the outer edge to thereby open the first cavity and define a second cavity receiving both of the first and second opposing portions of the outer edge thereinto with the absorbent article so folded, whereby the retainer flap retains the first and second opposing portions of the outer edge in the second cavity, and wherein a mid-section of the absorbent article optionally extends outwardly from the second cavity and defines a portion of an outer surface of the absorbent article as so folded.

In preferred embodiments, the retainer flap is resiliently extensible and resilient forces actively retract the retainer flap about the first and second opposing portions of the outer edge at the second cavity after the second cavity is fully formed with the first and second opposing portions of the outer edge in the second cavity.

Also in preferred embodiments, a first cavity portion of the absorbent article underlying the retainer flap at the first cavity is configured such that the absorbent is retained between the baffle and the cover after the second cavity is formed, optionally such that the baffle forms a corresponding portion of the surface opposing the body-facing surface after the second cavity is formed.

In some embodiments, the surface opposed to the body-facing surface is defined in part by the retainer flap and in part by the baffle.

In preferred embodiments, the absorbent comprises superabsorbent polymer.

The invention further comprehends methods of hygienically preparing a labial pad for disposal. The labial pad has a body-facing surface and a surface opposing the body-facing surface. The method comprises obtaining a labial pad ready for disposal. The pad has a cover defining at least a portion of the body-facing surface, a baffle defining a portion of the surface opposing the body-facing surface, an absorbent between the cover and the baffle, and a retainer flap mounted in the labial pad and overlying a portion of the baffle such that the surface opposing the body-facing surface is defined in part by the retainer flap and in part by the baffle. The labial pad further has an outer edge defining a first outer perimeter of the labial pad. The retainer flap has a second outer perimeter including a first portion secured in the labial pad at or adjacent the first outer perimeter and a second portion extending across an outer surface of the baffle. The second portion of the second outer perimeter of the retainer flap is displaceable from the baffle thereby to define a first cavity between the retainer flap and the baffle. The method further comprises folding the labial pad and thereby folding the body-facing surface upon itself such that first and second opposing portions of the outer edge are brought into facing relationship to each other. The method also comprises folding the retainer flap over both of the folded over first and second opposing portions of the outer edge and thereby opening the first cavity and correspondingly defining a second cavity which receives thereinto both of the first and second facing opposing portions of the outer edge, with the labial pad in the folded condition, whereby the retainer flap assists in retaining the first and second facing opposing portions of the outer edge in the second cavity.

In preferred methods, a first cavity portion of the absorbent article underlying the retainer flap at the first cavity comprises the baffle forming a corresponding portion of the surface opposing the body-facing surface after the second cavity is formed.

Also in preferred methods, the retainer flap is resiliently extensible, and the method includes resiliently stretching the retainer flap while folding the retainer flap over the folded over first and second opposing portions of the outer edge, whereby the retainer flap is sized and configured such that residual resilient forces in the retainer flap actively retract the retainer flap about the first and second opposing portions of the outer edge at the second cavity after the second cavity is fully formed with the first and second opposing portions of the outer edge in the second cavity.

### BRIEF DESCRIPTION OF THE DRAWINGS

The foregoing and other features, aspects and advantages of the present invention will become better understood with regard to the following description, appended claims, and accompanying drawings where:

FIG. 1 shows a simplified anatomical cross-sectional view of a human female illustrating a labial pad of the invention disposed within the vestibule in a use position.

FIG. 2 is a simplified anatomical cross-sectional view of a human female illustrating the environment for a labial pad.

FIG. 3 shows a top view of a first embodiment of a labial pad of the invention.

FIG. 4 shows a cross-sectional view of the embodiment of FIG. 3 and taken at 4-4 of FIG. 3.

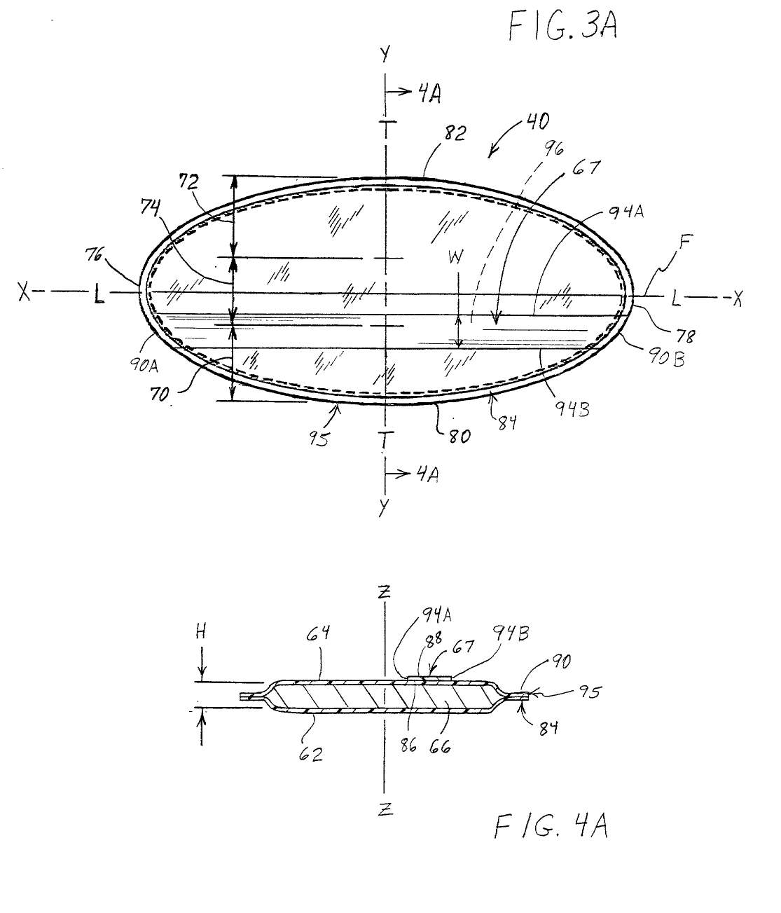

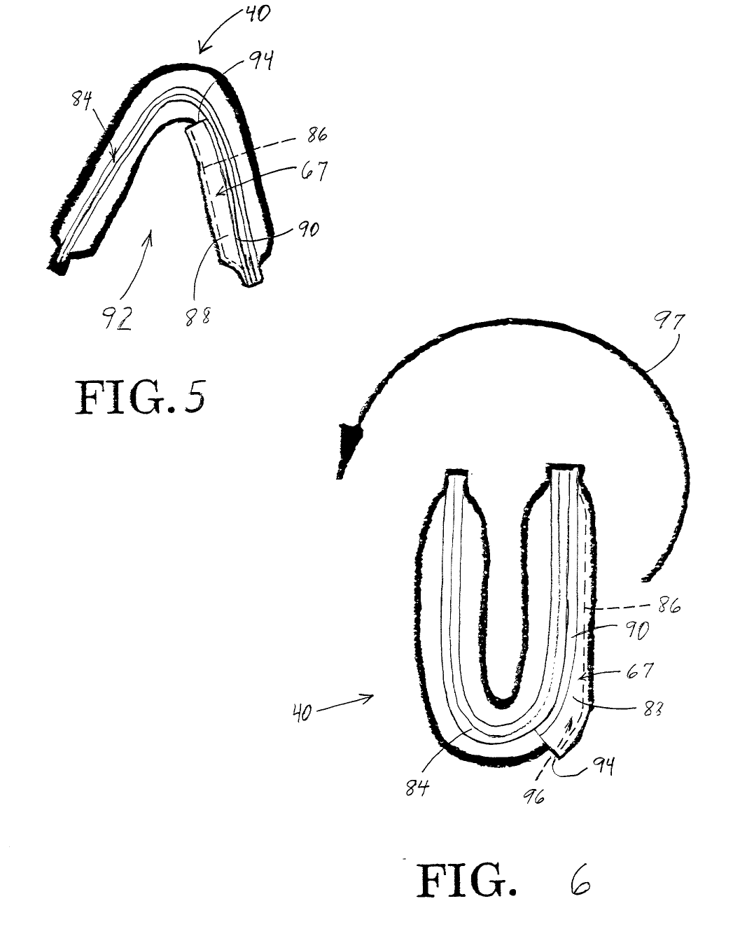

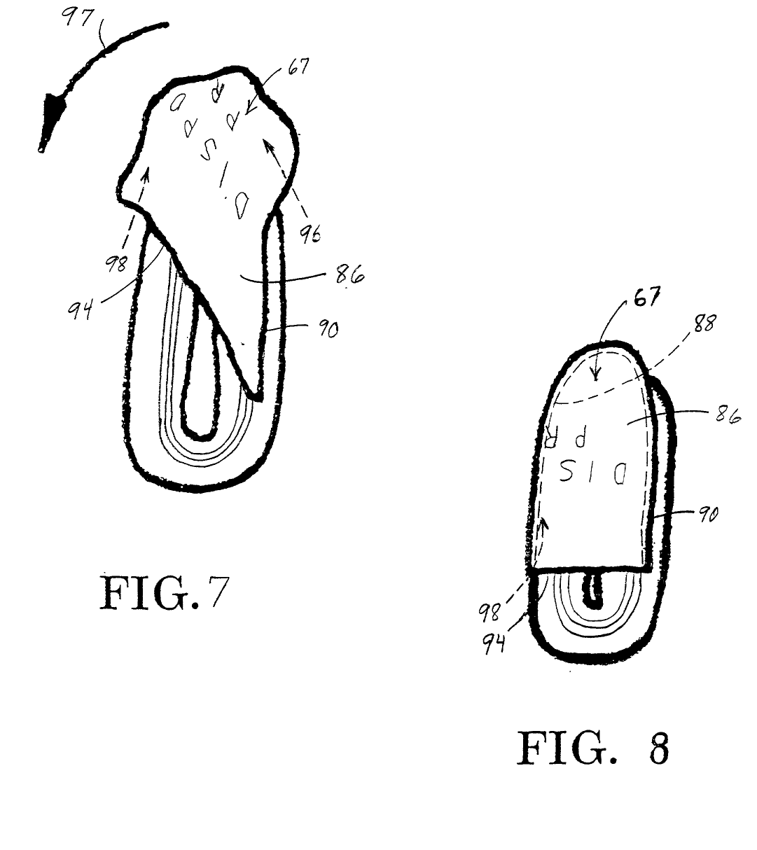

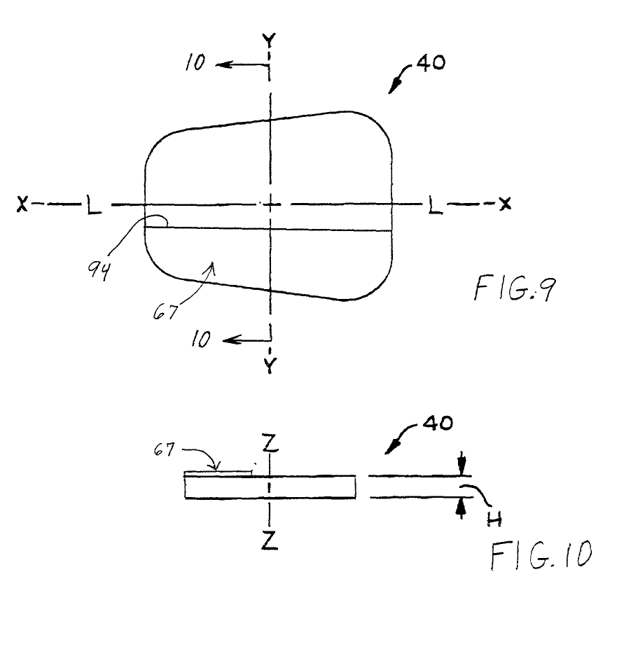

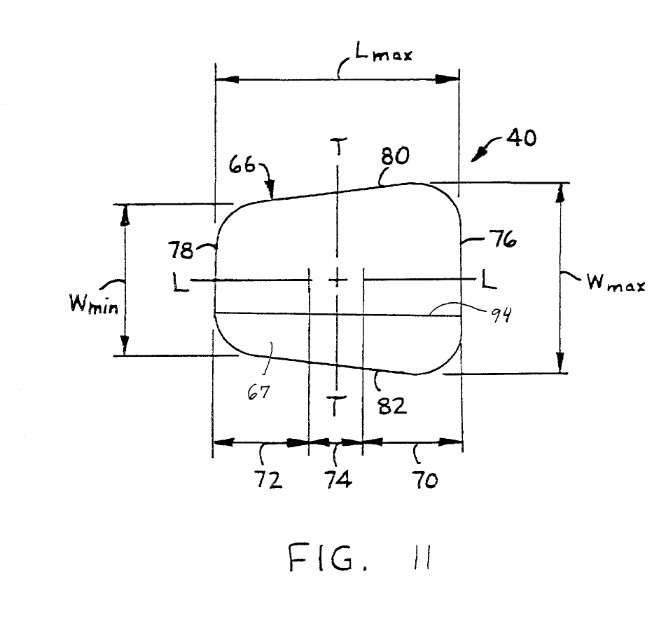

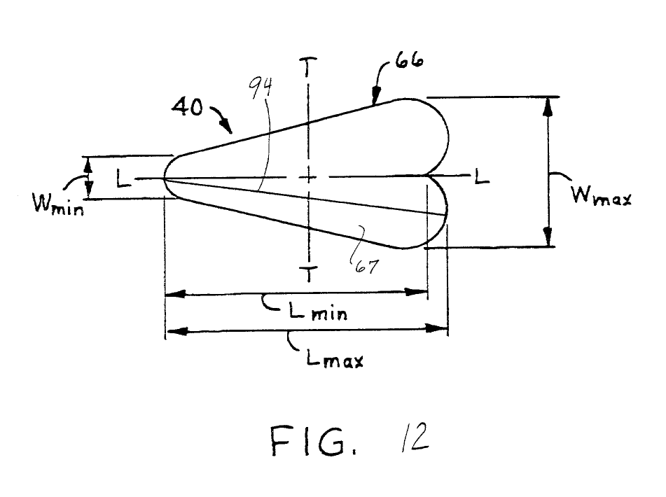

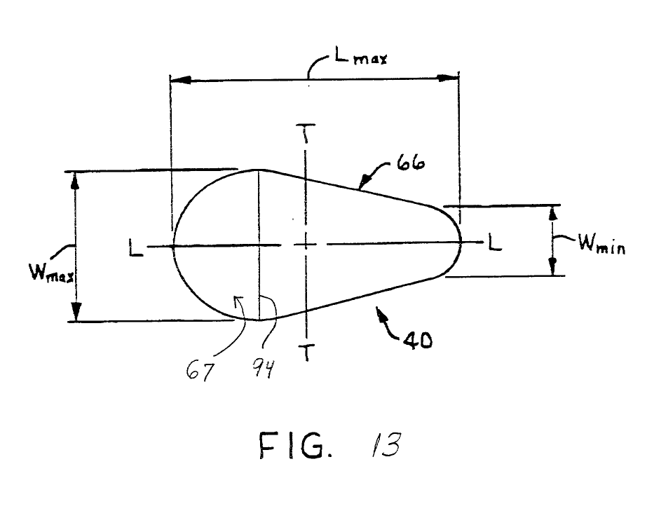

FIG. 3A shows a top view of the labial pad of FIG. 3, employing a modified version of the retainer flap.

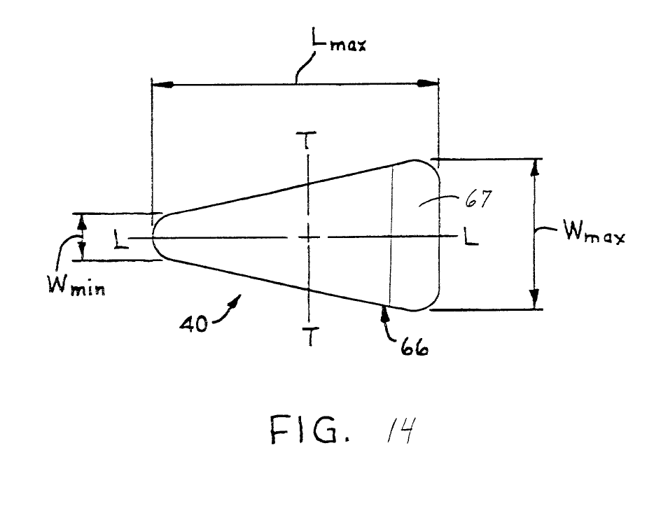

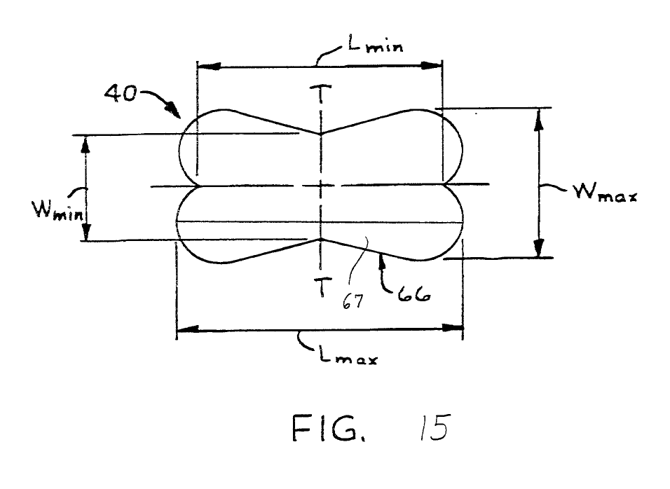

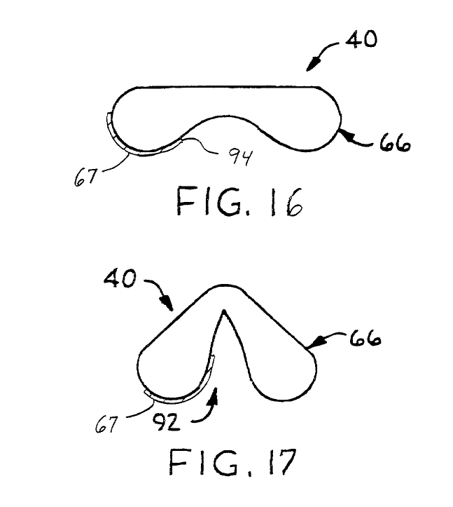

FIG. 4A shows a cross-sectional view of the embodiment of FIG. 3A and is taken at 4A-4A of FIG. 3A.

FIG. 5 shows a front elevation view of the labial pad of FIG. 3, inverted and folded substantially about a longitudinal axis thereof in preparation for positioning of the labial pad into the vestibule of a wearer.

FIG. 6 shows a front elevation view of the labial pad of FIG. 3 folded with the body-facing side of the pad disposed inwardly, and with opposing portions of the outer edge of the pad in facing relationship, as part of preparation for disposal of the pad.

FIG. 7 shows a front elevation view of the labial pad of FIG. 6 wherein the retainer flap is in the process of being folded over the opposing portions of the outer edge thereby to open the first cavity and create the second cavity, and thereby to retain the labial pad in the folded position with the opposing portions of the outer edge retained in facing relationship with each other in the second cavity.

FIG. 8 shows a front elevation view of the labial pad of FIG. 7 wherein the retainer flap has been fully folded over the opposing portions of the outer edge and accordingly retains the opposing portions of the outer edge in facing relationship, whereby the labial pad is retained in the folded configuration shown, with the retainer flap enclosing substantially the entirety of the body-facing surface, for disposal.

FIG. 9 shows a top view illustrating a second embodiment of labial pads of the invention wherein the dimensions of the absorbent are generally coincident with the dimensions of the absorbent article.

FIG. 10 shows cross-sectional view of the labial pad illustrated in FIG. 9 taken along line 10-10 of FIG. 9.

FIG. 11 shows a top view of a third embodiment of labial pads of the invention, similar to that illustrated in FIG. 9 wherein the dimensions of the absorbent are generally coincident with the dimensions of the absorbent article.

FIG. 12 shows a top view of a fourth embodiment of labial pads of the invention wherein the dimensions of the absorbent are generally coincident with the dimensions of the absorbent article.

FIG. 13 shows a top view a fifth embodiment of labial pads of the invention wherein the dimensions of the absorbent are generally coincident with the dimensions of the absorbent article.

FIG. 14 shows a top view of a sixth embodiment of labial pads of the invention wherein the dimensions of the absorbent are generally coincident with the dimensions of the absorbent article.

FIG. 15 shows a top view of a seventh embodiment of labial pads of the invention wherein the dimensions of the absorbent are generally coincident with the dimensions of the absorbent article.

FIG. 16 shows an end view of a eighth embodiment of labial pads of the invention wherein the dimensions of the absorbent are generally coincident with the dimensions of the absorbent article.

FIG. 17 shows an end view illustrating the embodiment of FIG. 16 in a folded configuration in preparation for insertion into the vestibule of a wearer.

The invention is not limited in its application to the details of construction or the arrangement of the components set forth in the following description or illustrated in the drawings. The invention is capable of other embodiments or of being practiced or carried out in other various ways. Also, it is to be understood that the terminology and phraseology employed herein is for purpose of description and illustration and should not be regarded as limiting. Like reference numerals are used to indicate like components.

### DESCRIPTION OF THE ILLUSTRATED EMBODIMENTS

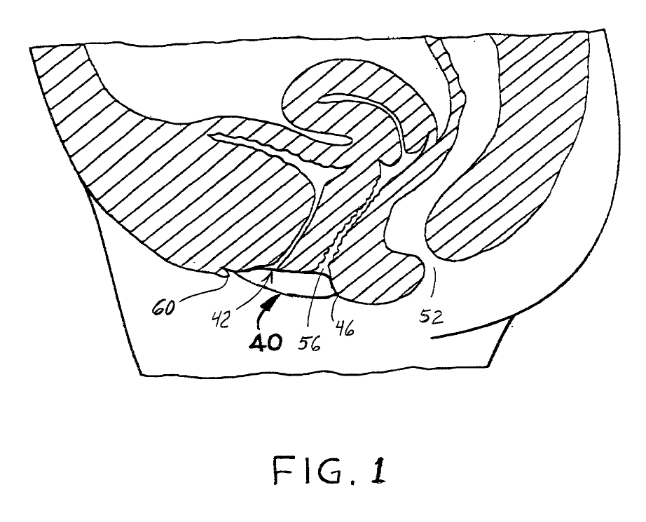

Referring to the drawings, in each of which similar parts are identified with like reference characters, FIG. 1 illustrates diagrammatically an absorbent article, such as a labial pad, designated generally as (40), disposed within the vestibule (42) of a wearer.

As used herein, the term “labial pad” refers to a device having at least some absorbent components, and which is specifically configured for disposition between the labia majora, extending at least partially into the vestibule (42) of a female wearer during use.

For purposes of this description, the vestibule (42) is considered to be the region defined within the labia (not specifically shown in the figures herein) beginning at about a point lying caudally from the anterior labial commissure indicated generally at (44), extending rearward to the posterior labial commissure (46) and bounded inwardly by the floor (48) of the vestibule.

One of skill in the art fully understands that there is a wide range of variation among women with respect to the relative size and shape of labia majora and labia minora as the same interrelatedly define the contour of the vestibule (42). For purposes of the present description, however, such differences will not specifically be addressed, it being recognized that in any event the disposition of the absorbent article (40) into the vestibule (42) necessitates placement between the labia majora regardless of any such consideration respecting the labia minora.

Lying caudally of the vestibule (42) is the perineum (50) which leads to the anus (52) in the region of the buttocks (54). Within the vestibule (42) itself are located the principal urogenital members which, for purposes pertinent here, are constituted of the vaginal orifice (56), the urethral orifice (58), and the clitoris (60). Given the foregoing simplified review of this anatomical region, and to facilitate the present description, the vestibule (42) is considered generally to be the region between the posterior labial commissure (46) and the clitoris (60). For a more comprehensive description of this portion of the human female anatomy, attention is invited to Anatomy of the Human Body by Henry Gray, Thirtieth American Edition (Carmine D. Clemente ed., Lea & Febiger, 1985) at 1571-1581.

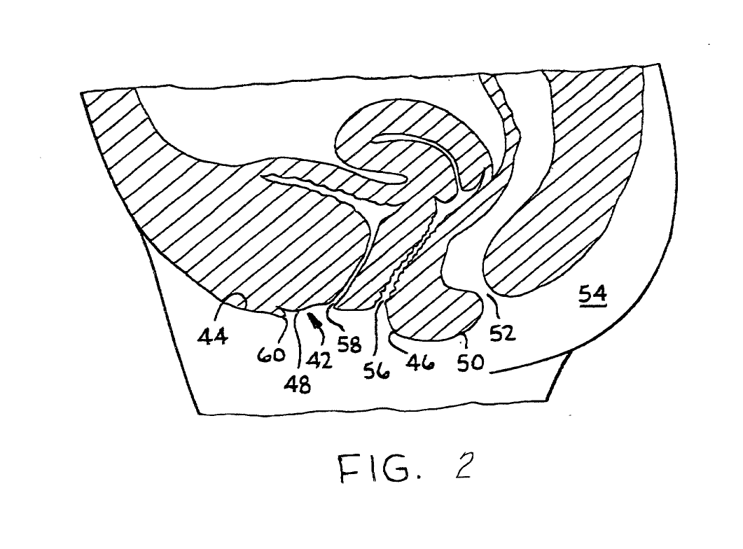

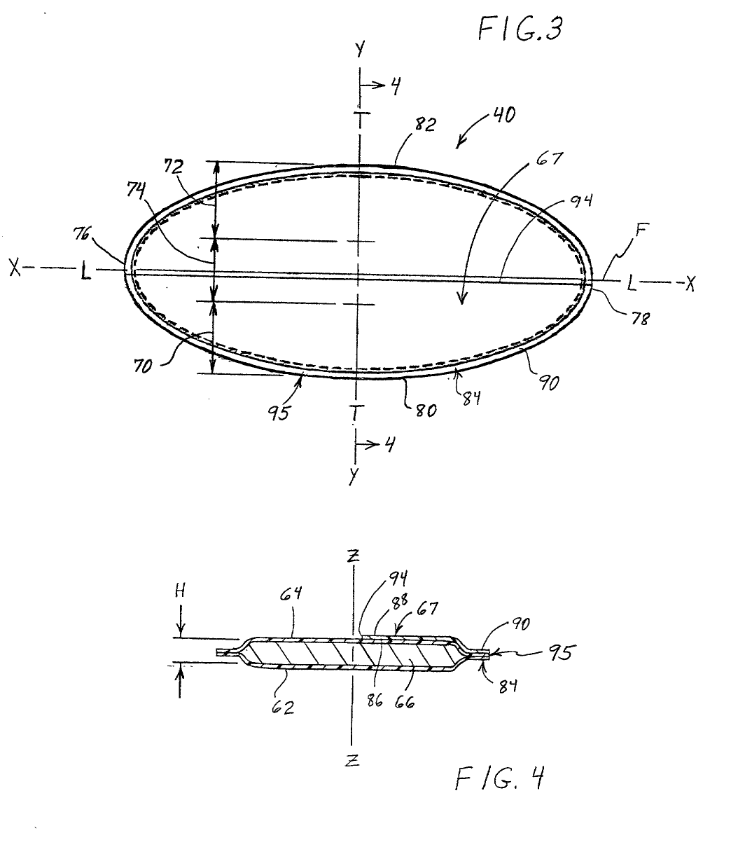

As can be seen with reference to the anatomical structure illustrated in FIGS. 1 and 2, the absorbent article (40) is disposed at least partially within the vestibule (42) for at least partially occluding the vestibule with respect to fluid flow from the vestibule. In this regard, the predominant use of the absorbent article (40) is for the absorption of menstrual fluid emitted via the vaginal orifice (56); although the absorbent article is equally well adapted to serve as a type of incontinence device for absorption of urine as occurs upon minor, female incontinence.

The absorbent article (40), a first embodiment of which is illustrated in FIGS. 3-8, has a principal longitudinal axis (L) which generally runs along the x direction. As used herein, the term “longitudinal” refers to a line, axis or direction in the plane of the absorbent article (40) which is generally aligned with (e.g., approximately parallel to) a vertical plane which bisects a standing female wearer into left and right body halves when the absorbent article is in use. The longitudinal direction is generally illustrated in FIGS. 3 and 9 by the x-axis.

The absorbent article (40) also has a transverse axis (T). The terms “transverse,” “lateral” or “y direction” as used herein generally refer to a line, axis or direction which is generally perpendicular to the longitudinal direction and in a surface defined between a body-facing surface of the pad and a surface opposing the body-facing surface. The lateral direction is generally illustrated in FIGS. 3 and 9 by the y-axis.

The “z direction” is typically a line, axis or direction generally parallel to the vertical plane described above. The z direction is generally illustrated in FIGS. 4 and 10 by the indicated z-axis.

The terms “upper” or upwardly refer generally to an orientation directed toward the wearer's head, while the terms “lower” or “downwardly” refer generally to an orientation directed toward the wearer's feet. For purposes of discussion herein, each layer of the absorbent article (40), e.g., a fluid permeable cover (62), a liquid impermeable baffle (64), an absorbent (66), and/or a retainer flap (67) has an upper or body-facing surface and a lower surface also described as the surface opposed to the upper or body-facing surface. Cover (62), baffle (64), absorbent (66), and retainer flap (67) are shown in e.g. FIG. 4.

Turning now to FIGS. 3 and 4 specifically, an absorbent article (40) is illustrated as including liquid permeable cover (62), liquid impermeable baffle (64) absorbent (66) situated between the cover and the baffle, and retainer flap (67) overlying a portion of the baffle. As illustrated in FIG. 3, and referencing the minor axis Y-Y, the absorbent (66) has a first end or side section (70), a second end or side section (72), and a mid-section (74) disposed between the end or side sections. Referring to FIG. 11, the end or side section (70) and the end or side section (72) are shown oriented along the major axis X-X. Generally, the end or side sections are defined for the purpose of illustrating orientation of the retainer flap (67). Thus, the end or side sections can be oriented in any desired direction.

The absorbent article (40) should be of a suitable size and shape which allows at least a portion, preferably a major portion, of the absorbent article to be disposed within the vestibule (42) of a female wearer. In addition, the absorbent article (40) desirably at least partially occludes and intercepts the flow of menstrual fluid, urine or other bodily exudates from the wearer's vaginal orifice (56) and/or urethral orifice (58).

The absorbent (66), and thus the absorbent article (40), generally displays a geometry extending between spaced apart first (76) and second (78) transverse end edges. The overall geometry is completed by noting that the absorbent (66), and thus the absorbent article (40), also includes spaced apart first (80) and second (82) longitudinal side edges extending between the transverse end edges (76, 78). The side and end edges can merge with each other at e.g. corners of the absorbent article as illustrated in FIG. 11, or can be part of an overall curvilinear outer perimeter configuration as illustrated in FIG. 3. The side edges and end edges collectively define the outer perimeter of the labial device.

The geometry of the absorbent (66) is a significant factor affecting the overall size, shape, and effectiveness of the absorbent article (40). In general, the absorbent (66) has a maximum width (Wₘₐₓ), and a minimum width (Wₘᵢₙ) as illustrated in e.g. FIG. 12. The maximum width (Wₘₐₓ) is measured along a line lying generally parallel to the principle transverse axis (T) and running from one longitudinal side to the opposing longitudinal side (80, 82). The a minimum width (Wₘᵢₙ) is measured along a line also lying generally parallel to the principal transverse axis (T) and running from one longitudinal side to the opposing longitudinal side (80, 82). The maximum width (Wₘₐₓ) of the absorbent (66) typically is no greater than about 30 mm; alternatively, no greater than about 40 mm; alternatively, no greater than about 50 mm; alternatively, no greater than about 60 mm; or alternatively, no greater than about 70 mm. The minimum width (Wₘᵢₙ) of the absorbent (66) typically is no less than about 30 mm; alternatively, no less than about 20 mm; alternatively, no less than about 10 mm; or alternatively, no less than about 5 mm. Thus, the absorbent (66) can have a width ranging from no less than about 5 mm up to no greater than about 70 mm; although the approximate width(s) of the absorbent can vary according to, inter alia, the general design and intended disposition of the absorbent article (40) within the vestibule (42) of a female wearer. One of skill in the art will readily appreciate that certain versions of the absorbent (66), and thus certain versions of the absorbent article (40), can have a minimum width (Wₘᵢₙ) equal to its maximum width (Wₘₐₓ). In such instances, reference is generally made only to the maximum width (Wₘₐₓ).

The absorbent (66) also has a maximum length (Lₘₐₓ), measured along a line lying generally parallel to the principal longitudinal axis (L) and running from one transverse end edge to the other transverse end edge (76, 78). The maximum length (Lₘₐₓ) of the absorbent (66) typically is no greater than about 40 mm; alternatively, no greater than about 50 mm; alternatively, no greater than about 60 mm; alternatively, no greater than about 70 mm; alternatively, no greater than about 80 mm; alternatively, no greater than about 90 mm; or alternatively, no greater than about 100 mm.

The absorbent (66) can also have a minimum length (Lₘᵢₙ), measured along a line also lying generally parallel to principle longitudinal axis (L) and running from one transverse end edge to the other transverse end edge (76, 78). The minimum length (Lₘᵢₙ) of the absorbent (66) typically is no less than about 100 mm; alternatively, no less than about 90 mm; alternatively, no less than about 80 mm; alternatively, no less than about 70 mm; alternatively, no less than about 60 mm; alternatively, no less than about 50 mm; or alternatively, no less than about 40 mm. Thus, the absorbent (66) can have a length ranging from no less than about 40 mm up to no greater than about 100 mm; although the approximate length(s) of the absorbent can vary according to, inter alia, the general design and intended disposition of the absorbent article (40) within the vestibule (42) of a female wearer.

One of skill in the art will readily appreciate that certain versions of the absorbent (66), and thus certain versions of the absorbent article (40), can have a minimum length (Lₘᵢₙ) equal to the maximum length (Lₘₐₓ) In such instances, as illustrated, for example, in FIG. 11, reference is generally made only to the maximum length (Lₘₐₓ). Embodiments of an absorbent (66), and thus embodiments of an absorbent article (40), having a maximum length (Lₘₐₓ) not equal to the minimum length (Lₘᵢₙ) are illustrated, for example, in FIGS. 12 and 15.

The absorbent article (40) is desirably provided with sufficient capacity to absorb and retain the intended amount and type of bodily exudate(s). The absorbent capacity is provided by a fluid retentive core or absorbent generally identified as (66). For at least menstrual fluid, the absorbent (66) desirably has a minimum capacity of no less than about 19 g/g; alternatively, no less than about 18 g/g; alternatively, no less than about 17 g/g; alternatively, no less than about 16 g/g; alternatively, no less than about 15 g/g; alternatively, no less than about 14 g/g; alternatively, no less than about 13 g/g; alternatively, no less than about 12 g/g; alternatively, no less than about 11 g/g; alternatively, no less than about 10 g/g; alternatively, no less than about 9 g/g; alternatively, no less than about 8 g/g; alternatively, no less than about 7 g/g; alternatively, no less than about 6 g/g; alternatively, no less than about 5 g/g; alternatively, no less than about 4 g/g; alternatively, no less than about 3 g/g; alternatively, no less than about 2 g/g; or alternatively, no less than about 1 g/g.

The absorbent (66) also can have a maximum capacity of no greater than about 5 g/g; alternatively, no greater than about 6 g/g; alternatively, no greater than about 7 g/g; alternatively, no greater than about 8 g/g; alternatively, no greater than about 9 g/g; alternatively, no greater than about 10 g/g; alternatively, no greater than about 11 g/g; alternatively, no greater than about 12 g/g; alternatively, no greater than about 13 g/g; alternatively, no greater than about 14 g/g; alternatively, no greater than about 15 g/g; alternatively, no greater than about 16 g/g; alternatively, no greater than about 17 g/g; alternatively, no greater than about 18 g/g; alternatively, no greater than about 19 g/g; alternatively, no greater than about 20 g/g; alternatively, no greater than about 25 g/g; or alternatively, no greater than about 30 g/g. Thus, the absorbent (66) can have an absorbent capacity ranging from no less than about 1 g/g up to no greater than about 30 g/g; although the approximate capacity of the absorbent can vary according to, inter alia, the general design and intended disposition of the absorbent article (40) within the vestibule (42) of a female wearer. One of skill in the art will readily realize that the addition of superabsorbent polymer or coated superabsorbent polymer) to the absorbent (66) typically has the effect of substantially increasing the absorbent capacity.

Describing the individual elements in greater detail, the absorbent (66) has an upper or body-facing surface and a lower surface, or surface opposed to the upper or body-facing surface, and can include any material capable of absorbing and/or adsorbing and thereafter retaining the intended bodily exudate(s). Suitable materials are also generally hydrophilic, compressible and conformable.

The absorbent (66) can be formed from any of the materials well known to those of ordinary skill in the art. Examples of such materials include, but are not limited to, various natural or synthetic fibers, multiple plies of creped cellulose wadding, fluffed cellulose fibers, rayon or other regenerated cellulose materials, wood pulp fibers or comminuted wood pulp fibers, airlaid material, textile fibers, a blend of polyester fibers and polypropylene fibers, absorbent foams, absorbent sponges, superabsorbent polymers, coated superabsorbent polymers, fibrous bundles or nits, or any equivalent material or combination of materials. Hydrophobic materials are also suitable for use where the hydrophobic material has been rendered hydrophilic according to any of a number of known methods for so doing.

The total absorbent capacity of the absorbent (66) should, however, be compatible with the designed exudate loading and the intended use of the absorbent article (40). Further, the size and absorbent capacity of the absorbent (66) can be varied. Therefore, the dimension, shape, and configuration of the absorbent (66) can be varied. For example, the absorbent can have a varying thickness as illustrated at least in FIGS. 16 and 17, or can have an hydrophilic gradient, or can contain superabsorbent polymer(s) and the like.

The absorbent (66) generally has a thickness, caliper or height (H), as illustrated in e.g. FIG. 4, measured along a line lying generally parallel to the z-axis. The minimum thickness of the absorbent (66) typically is no less than about 9 mm; alternatively, no less than about 8 mm; alternatively, no less than about 7 mm; alternatively, no less than about 6 mm; alternatively, no less than about 5 mm; alternatively, no less than about 4 mm; alternatively, no less than about 3 mm; alternatively, no less than about 2 mm; or alternatively, no less than about 1 mm. The maximum thickness of the absorbent (66) typically is no greater than about 2 mm; alternatively, no greater than about 3 mm; alternatively, no greater than about 4 mm; alternatively, no greater than about 5 mm; alternatively, no greater than about 6 mm; alternatively, no greater than about 7 mm; alternatively, no greater than about 8 mm; alternatively, no greater than about 9 mm; or alternatively, no greater than about 10 mm. Thus, the absorbent (66) can have a thickness of about 10 mm or less; although the approximate thickness of the absorbent can vary according to, inter alia, the general design and intended disposition of the absorbent article (40) within the vestibule (42) of a female wearer.

The absorbent (66) desirably also has a relatively low density which is desirable for comfort. Generally, the absorbent has a density of less than about 0.5 g/cc. Stated differently, the absorbent (66) typically has a maximum density of no greater than about 0.5 g/cc; alternatively, no greater than about 0.4 g/cc; alternatively, no greater than about 0.3 g/cc; alternatively, no greater than about 0.2 g/cc; alternatively, no greater than about 0.1 g/cc; alternatively, no greater than about 0.09 g/cc; alternatively, no greater than about 0.08 g/cc; alternatively, no greater than about 0.07 g/cc; alternatively, no greater than about 0.06 g/cc; alternatively, no greater than about 0.05 g/cc; alternatively, no greater than about 0.04 g/cc; alternatively, no greater than about 0.03 g/cc; or alternatively, no greater than about 0.02 g/cc.

The absorbent (66) generally also has a minimum density of typically no less than about 0.01 g/cc; alternatively no less than about 0.02 g/cc; alternatively, no less than about 0.03 g/cc; alternatively, no less than about 0.04 g/cc; alternatively, no less than about 0.05 g/cc; alternatively, no less than about 0.06 g/cc; alternatively, no less than about 0.07 g/cc; alternatively, no less than about 0.08 g/cc; alternatively, no less than about 0.09 g/cc; alternatively, no less than about 0.1 g/cc; alternatively, no less than about 0.2 g/cc; alternatively, no less than about 0.3 g/cc; or alternatively, no less than about 0.4 g/cc. Thus, the density of the absorbent (66) can range up to about 0.5 g/cc; although the approximate density of the absorbent can vary according to, inter alia, the general design and intended disposition of the absorbent article (40) within the vestibule (42) of a female wearer.

The absorbent (66) also desirably has a basis weight of less than about 600 grams per square meter (gsm). Stated differently, the absorbent (66) typically has a maximum basis weight of no greater than about 600 gsm; alternatively, no greater than about 500 gsm; alternatively, no greater than about 400 gsm; alternatively, no greater than about 300 gsm; alternatively, no greater than about 200 gsm; or alternatively, no greater than about 100 gsm.

Generally, the absorbent (66) also has a minimum basis weight of typically no less than about 0.1 gsm; alternatively, no less than about 1 gsm; alternatively, no less than about 50 gsm; alternatively, no less than about 100 gsm; alternatively, no less than about 150 gsm; alternatively, no less than about 200 gsm; alternatively, no less than about 250 gsm; alternatively, no less than about 300 gsm; alternatively, no less than about 350 gsm; alternatively, no less than about 400 gsm; alternatively, no less than about 450 gsm; alternatively, no less than about 500 gsm; or alternatively, no less than about 550 gsm. Thus, the absorbent (66) can have a basis weight of about 600 gsm or less; although the approximate basis weight of the absorbent can vary according to, inter alia, the general design and intended disposition of the absorbent article (40) within the vestibule (42) of a female wearer. A specific example of a suitable absorbent is a coform material made of a blend of polypropylene and cellulose fibers such as that used in KOTEX maxi pantiliners and obtainable from Kimberly-Clark Corporation, Neenah, Wis., USA.

The baffle (64) typically resides on the lower surface of the absorbent (66) as the absorbent article is worn by a wearer, and can be constructed from any desired material which is liquid-impermeable. Desirably, the baffle (64) permits passage of air and moisture vapor out of the absorbent (66), while blocking passage of bodily fluid(s). An example of a suitable baffle material is a micro-embossed, polymeric film, such as polyethylene, polypropylene or polyester, having a minimum thickness of no less than about 0.025 mm and a maximum thickness of no greater than about 0.13 mm. Bicomponent films can also be used, as well as woven and nonwoven fabrics which have been treated to render such fabrics liquid-impermeable. An example of another suitable material is a closed cell polyolefin foam, for example, a closed cell polyethylene foam.

The baffle (64) can be maintained in secured relation with the absorbent (66) by bonding all or a portion of the adjacent surfaces to one another. A variety of bonding methods known to one of skill in the art can be utilized to achieve any such secured relation. Examples of such methods include, but are not limited to, ultrasonic bonding, thermal bonding, or the application of adhesive materials in a variety of patterns between the two adjoining surfaces. A specific example of a baffle material is a polyethylene film such as that used in KOTEX pantiliners and obtainable from Pliant Corporation, Schaumburg, Ill., USA.

Optional fluid permeable cover (62) has an upper surface and a lower surface as the absorbent article is worn by a wearer, with the upper surface typically contacting the body of the wearer and receiving bodily exudate(s). The cover (62) desirably is made of a material which is flexible and non-irritating to the tissues within the vestibule (42) of a female wearer. As used herein, the term “flexible” is intended to refer to materials which are compliant and readily conform to the bodily surface(s) with which such materials are in contact, or materials which respond by easily deforming in the presence of external forces.

The cover (62) is provided for comfort and conformability and functions to direct bodily exudate(s) away from the body, through the cover (62) and toward the absorbent (66). The cover (62) should retain little or no liquid in its structure so that the cover provides a relatively comfortable and non-irritating surface next to the tissues within the vestibule (42) of a female wearer. The cover (62) can be constructed of any woven or nonwoven material which is easily penetrated by bodily fluids which contact the surface of the cover. Examples of suitable cover materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. A specific example of a suitable cover material is a bonded carded web made of polypropylene and polyethylene such as that used as cover stock for KOTEX pantiliners and obtainable from Sandler Corporation, Germany. Other examples of suitable materials are composite materials of polymer and nonwoven fabric materials. The composite materials are typically in the form of integral sheets generally formed by the extrusion of a polymer onto a web of spunbonded material. The fluid permeable cover (62) can also contain a plurality of apertures (not shown) formed therein which are intended to increase the rate at which bodily fluid(s) can penetrate through the cover and into the absorbent (66).

A physiologically hydrous cover material is also suitable for use. As used herein, the phrase “physiologically hydrous” is intended to connote a cover material which maintains a suitably moist interface between the tissues of the vestibule (42) and the absorbent article (40) when disposed in the vestibular environment; material which is benign respecting the requirements of comfort associated with the interposition of fabric or fabric-like structures within the moist tissue environment of the vestibule, also considering that the absorbent article receives bodily fluid(s) migrating through the vestibule and conducts such fluids to the absorbent (66). Thus, while the cover (62) is not “hydrous” in the classic sense prior to use, inasmuch as the cover is dry at that time, the cover (62) maintains, or at least does not interfere with the maintenance of, the proper moisture level or moisture balance required within the vestibule (42) for proper maintenance of tissue health within the vestibule.

At least a portion of the surface of the cover (62) can be treated with a surfactant in order to render the cover more hydrophilic. This results in permitting the insulting bodily fluid(s) to more readily penetrate the cover (62). The surfactant can also diminish the likelihood that the insulting bodily fluid(s), such as menstrual fluid, will flow off the cover (62) rather than passing through the cover and being absorbed by the absorbent (66). One suitable approach provides for the surfactant to be substantially evenly distributed across at least a portion of the upper surface of the cover (62) which overlies the upper surface of the absorbent (66).

The cover (62) can be maintained in secured relation with the absorbent (66) by bonding all or a portion of the adjacent surfaces to one another. A variety of bonding methods known to one of skill in the art can be utilized to achieve any such secured relationship. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover (62) typically resides on the upper surface of the absorbent (66), but alternatively can surround and partially or entirely enclose the absorbent. Alternatively, the cover (62) and the baffle (64) can have peripheries which extend outward beyond the periphery of the absorbent (66) and can be peripherally joined together to form an edge (84), as illustrated in FIGS. 3 and 4. Utilizing known techniques, such as, for example, gluing, crimping, hot-sealing or the like, the edge (84) can be formed either entirely, so that the entire periphery of the absorbent (66) is circumscribed by their joinder, as in FIGS. 3 and 4, or the cover (62) and the baffle (64) can be partially peripherally joined.

To minimize the possibility of irritation and/or discomfort to the wearer of the absorbent article (40), it is desired that the edge (84) and at least the area of the absorbent article immediately adjacent the edge be soft, compressible and conformable. Desirably, any edge (84) so formed has a width no greater than about 10 mm; alternatively, no greater than about 9 mm; alternatively, no greater than about 8 mm; alternatively, no greater than about 7 mm; alternatively, no greater than about 6 mm; alternatively, no greater than about 5 mm; alternatively, no greater than about 4 mm; alternatively, no greater than about 3 mm; alternatively, no greater than about 2 mm; or alternatively, no greater than about 1 mm. In addition, any edge (84) so formed desirably has a width of no less than about 0.5 mm; alternatively, no less than about 1 mm; alternatively, no less than about 2 mm; alternatively, no less than about 3 mm; alternatively, no less than about 4 mm; alternatively, no less than about 5 mm; alternatively, no less than about 6 mm; alternatively, no less than about 7 mm; alternatively, no less than about 8 mm; or alternatively, no less than about 9 mm. Thus, any edge (84) so formed can have a width ranging from no less than about 0.5 mm up to no greater than about 10 mm; although the approximate width of any edge can vary according to, inter alia, the general design and intended disposition of the absorbent article (40) within the vestibule (42) of a female wearer. In other versions, the cover (62) and/or the baffle (64) and/or the retainer flap (67) can have a periphery which is coterminous with the periphery of the absorbent (66).

Positioned either on or substantially parallel to the principal longitudinal axis (L) of the absorbent (66), is, optionally, a desired axis of flexure (F) as illustrated in FIG. 3. A desired axis of flexure (F) typically runs in the longitudinal direction, i.e., along the x direction, and may be off center from the principle longitudinal axis (L) a distance of no greater than about 10 mm; alternatively, no greater than about 9 mm; alternatively, no greater than about 8 mm; alternatively, no greater than about 7 mm; alternatively, no greater than about 6 mm; alternatively, no greater than about 5 mm; alternatively, no greater than about 4 mm; alternatively, no greater than about 3 mm; alternatively, no greater than about 2 mm; or alternatively, no greater than about 1 mm. Desirably, a desired axis of flexure (F) is aligned along the principal longitudinal axis (L) as illustrated in FIG. 3.

A desired axis of flexure (F) typically minimally extends longitudinally no less than about 90%; alternatively, no less than about 80%; alternatively, no less than about 70%; alternatively, no less than about 60%; alternatively, no less than about 50%; or alternatively, no less than about 40% of the maximum length (Lₘₐₓ) of the absorbent (66).

A desired axis of flexure (F) can result naturally from the dimensions, shape, and/or configuration of the absorbent (66), or the absorbent can be imparted with a weakened axis or region to create a desired axis of flexure. A desired axis of flexure (F) can also be formed by any of the techniques known to one of skill in the art, including, for example, scoring, pre-folding, slitting, embossing, or the like. Although a desired axis of flexure (F) is described herein as residing in the absorbent (66), one of skill in the art will readily appreciate that a desired axis of flexure can be formed in the cover (62), the baffle (64) and/or the absorbent; namely the cover and the baffle; the cover and the absorbent; or the baffle and the absorbent; or cover, baffle, and absorbent. The flexure axis (F) can as well be formed, in whole or in part, in the retainer flap (67). When present, a desired axis of flexure (F) typically facilitates folding of an absorbent article (40) prior to disposition within the vestibule (42) of a female wearer and/or folding for disposal of such absorbent article after use.

The thickness, caliper or height (H), as illustrated at least in FIGS. 4 and 10, is measured along a line lying generally parallel to the z-axis. The minimum thickness of the absorbent article (40), including retainer flap (67), typically is no less than about 9 mm; alternatively, no less than about 8 mm; alternatively, no less than about 7 mm; alternatively, no less than about 6 mm; alternatively, no less than about 5 mm; alternatively, no less than about 4 mm; alternatively, no less than about 3 mm; alternatively, no less than about 2 mm; alternatively, no less than about 1 mm; or alternatively, no less than about 0.5 mm.

The maximum thickness of the absorbent article (40), including retainer flap (67), typically is no greater than about 1 mm; alternatively, no greater than about 2 mm; alternatively, no greater than about 3 mm; alternatively, no greater than about 4 mm; alternatively, no greater than about 5 mm; alternatively, no greater than about 6 mm; alternatively, no greater than about 7 mm; alternatively, no greater than about 8 mm; alternatively, no greater than about 9 mm; or alternatively, no greater than about 10 mm. Thus, the absorbent article (40) can have a thickness of about 10 mm or less; although the approximate thickness of the absorbent article can vary according to, inter alia, the general design and intended disposition of the absorbent article within the vestibule (42) of a female wearer.

The absorbent article (40) typically is folded along an axis lying on or positioned parallel to the principle longitudinal axis (L), as illustrated at least in FIGS. 5 and 17, prior to disposition within the vestibule (42) of the female wearer. When folded along such longitudinal axis, the absorbent article (40) forms a recess (92) which generally represents the configuration of the absorbent article when the absorbent article is placed into the vestibule (42). Once inserted, the absorbent article (40) can have a tendency to unfold in an attempt to fill the vestibule and thus maintain the upper surface of the absorbent article in contact with the tissues of the vestibule (42). The absorbent article (40) can be resiliently biased along the axis about which the absorbent article is folded to increase the tendency of the absorbent article to unfold. Alternatively, the absorbent (66) of the absorbent article (40) can be thicker along its longitudinal edges, as illustrated in FIGS. 16 and 17, thereby to provide a biasing effect which causes the upper surface of the absorbent article (40) to contact the tissues of the vestibule (42).

An absorbent article (40) as described herein, however, does not necessarily require any additional features to maintain contact with the tissues of the vestibule (42) of the female wearer, as the naturally moist surfaces of the tissues of the vestibule (42) typically demonstrate a tendency to maintain contact with the upper surface of the absorbent article (40).

Retainer flap (67) has an upper surface (86) and a lower surface (88) as the article is worn by a wearer. The retainer flap (67) typically is positioned on the lower surface of the baffle (64) and is preferably biased into surface-to-surface relationship against the baffle (64). Retainer flap (67) is desirably constructed from material which is liquid-impermeable, and which permits passage of air and moisture vapor out of the absorbent, while blocking passage of bodily fluids.

Preferred materials for use in the retainer flap (67) are any of the flexible materials suitable for use in the baffle (64). In preferred embodiments, the retainer flap (67) can be a folded over extension of the material used in forming the baffle (64).

Thus, an example of suitable retainer flap material is a micro-embossed, polymeric film, such as polyethylene, polypropylene, or polyester, having a minimum thickness of no less than about 0.13 mm. Bicomponent films can also be used, as well as woven and nonwoven fabrics. An example of another suitable material is a closed cell polyolefin foam, for example, a closed cell polyethylene foam.

Retainer flap (67) preferably has at least a modest degree of resilient extensibility, e.g. at least about 20 percent, preferably at least about 25 percent extensibility, for reasons discussed hereinafter. In general, the materials recited above exhibit the desired degree of resilient extensibility. As desired, retainer flap material can be rendered resiliently extensible e.g. by gathering such material and securing thereto resiliently extensible elements such as threads of lycra to thereby render such materials resiliently extensible.

As used herein, resilient extensibility refers to a property of a material whereby the material can be extended and, when the extending force is removed, the material returns to substantially the length of the material before such extension. Return of at least 50 percent, preferably at least 75 percent, of the extension amount is considered a return to substantially the length of the material before such extension.

The retainer flap (67) can be maintained in secured relation with the baffle (64) and the absorbent (66) by bonding adjacent surfaces of a first portion (90) of the outer perimeter of the retainer flap at or adjacent, e.g. proximate, an outer perimeter of the absorbent article to either or both of facing surfaces of the baffle (64) or the absorbent (66), whereby the bonded first portion of the outer perimeter of the retainer flap can be part of the outer edge (84) of the absorbent article (40). A variety of bonding methods known to those of skill in the art can be utilized to achieve any such secured relation of the retainer flap to the absorbent article proximate the outer perimeter of the absorbent article. Examples of such bond methods include, but are not limited to, ultrasonic bonding, thermal bonding, or application of adhesive materials to one or both of the surfaces to be bonded together. A specific example of a retainer flap material is a polyethylene film such as that used in KOTEX pantiliners and obtainable from Pliant Corporation, Schaumburg, Ill., USA.

The first portion (90) of the outer perimeter of the retainer flap (67) is generally that portion of the perimeter of the retainer flap which corresponds with, or generally follows along, the outer perimeter (95) of the absorbent article (40). Preferably, the entirety of the first portion (90) of the outer perimeter of the retainer flap (67) is attached to the facing surfaces of one or more other elements of the absorbent article. In the alternative, the first portion can be intermittently attached to the one or more other elements.

The second portion (94) of the outer perimeter of the retainer flap (67) extends from the outer perimeter (95) of the absorbent article at a first locus across the lower surface of the absorbent article, preferably across the lower surface of the baffle (64), to a second locus of the outer perimeter (95) of the absorbent article. The second portion of the outer perimeter can extend parallel to central longitudinal axis (L), as in e.g. FIG. 3, or not parallel and thus transverse to the longitudinal axis (L), as in e.g. FIGS. 12 and 13.

FIGS. 3A and 4A illustrate another embodiment of the retainer flap (67) wherein the first portion (90) of the outer perimeter of the retainer flap is sectioned so as to have first (90A) and second (90B) sections thereof, spaced from each other along the outer perimeter (95) of the absorbent article, and representing first and second separate and distinct sections of the outer perimeter of the retainer flap. Accordingly, the second portion (94) of the outer perimeter of the retainer flap is also sectioned so as to have first (94A) and second (94B) sections thereof, spaced from each other across the width (W) of the retainer flap, and in FIGS. 3A and 4A across the width of the absorbent article, and representing third and fourth separate and distinct sections of the outer perimeter of the retainer flap. In FIGS. 3A and 4A, sections (94A) and (94B) are both spaced from the longitudinal side edge (80) of the absorbent article, whereby the first cavity (96) formed by the retainer flap (67) in FIGS. 3A and 4A is open, and thus open-ended, toward a major portion of side edge (80) of the absorbent article.

The first cavity (96) formed by the retainer flap (67) in FIGS. 3A and 4A performs all the desired functions for handling the absorbent article during placement in the vestibule. When the absorbent article (40) of FIGS. 3A and 4A is to be removed and disposed of after use in the vestibule, the retainer flap (67) can be manipulated by the user's hand as described above for the embodiment of FIGS. 3 and 4, and can also be manipulated in the folding-over function in preparation for disposal, thereby to hold the facing edge portions in facing relationship with each other. While the open-ended retainer flap (67) may not substantially enclose the entirety of the edge (84) of the absorbent article in the second cavity, some uses are contemplated where such degree of enclosure may not be necessary.

Thus, in the embodiment of FIGS. 3A and 4A, retainer flap (67) is embodied in a strip of material having a generally constant width (W), extending across a portion of the outer surface of the baffle (64), wherein both the first (90) and second (94) portions of the outer perimeter are segmented, and collectively represent the entirety of the outer perimeter of the retainer flap (67).

FIGS. 3A and 4A illustrate that the retainer flap (67) can be any width (W) so long as the width (W) is consistent with the functions of the user being able to use the retainer flap for holding the absorbent article while she places the absorbent article in the vestibule, and for retrieving the absorbent article from the vestibule. In addition, the width (W) can vary along the length of the retainer flap. In any event, the retainer flap must be consistent with the disposal preparation configurations illustrated in FIGS. 7 and 8, wherein the retainer flap holds the facing portions of the body-facing surface in facing relation with each other.

The edge of the retainer flap opposite section 94A of the first portion of the outer perimeter can be coincident with side edge (80) of the absorbent article as in e.g. FIGS. 3 and 4, or can be spaced from the side edge as in FIGS. 3A and 4A. Preferred embodiments are represented by e.g. FIGS. 3 and 4 because the configuration of the first portion of the outer perimeter, extending along the full length of the retainer flap along the side edge (80) of the absorbent article, provides a greater level of containment of any body exudates adjacent the folded-over first and second portions of the outer edge while the absorbent article is being prepared for disposal. Further, the second portion of the outer perimeter of the retainer flap, whether sectioned or not sectioned, need not be a straight line configuration, but can be any configuration consistent with the operations and functions described herein with respect to the retainer flap (67).

While the first portion of the outer perimeter of the retainer flap (67) is affixed, attached to one or more other elements of the absorbent article (40), the second portion of the outer perimeter is generally not affixed to any other element, e.g. baffle (64), of the absorbent article. In the alternative, the retainer flap (67) can be lightly affixed to another element of the absorbent article with a securement sufficiently weak that the affixation can be readily broken by a user of the absorbent article. Thus, the second portion (94) can be moved, displaced away from e.g. the baffle (64) so as to define a first cavity (96) between the retainer flap (67) and the baffle (64).

By so displacing the retainer flap (67) from the baffle (64), a user of the absorbent article (40) can insert her fingers into the first cavity (96) and thereby retain the absorbent article on her hand while she holds, handles, and places the absorbent article (40) in the vestibule (42) without touching the body-facing surface of the absorbent article (40). Similarly, for removing the used absorbent article (40) from the vestibule, the user can again insert her fingers into the first cavity and can so readily retain the absorbent article on her hand while she handles, moves, and removes, the absorbent article from the vestibule.

While the absorbent article (40) is in use in the vestibule (42), the absorbent article is folded similar to the configuration shown in FIG. 5. Once the used absorbent article (40) has been removed from the vestibule, the wearer can fold the body-facing surface of the absorbent article inwardly on itself e.g. about central longitudinal axis (L), to the configuration illustrated in FIG. 6. In the configuration shown in FIG. 6, the outer surface of the folded absorbent article is formed in part by the baffle (64) and in part by the retainer flap (67).

With the absorbent article in the configuration shown in FIG. 6, the user grasps the flexible material of the retainer flap (67) at the unaffixed second portion (94) of the perimeter of the flexible retainer flap material and pulls, flexes, and optionally stretches the unaffixed second portion up and over the facing opposing portions of the outer edge of the absorbent article, as illustrated and suggested by the arrows 97 in FIGS. 6 and 7, thus turning the retainer flap inside out. In so doing, the first cavity (96) is opened up. The retainer flap material at the second perimeter portion (94) is further pulled over the facing edge portions, and down on the opposing side of the absorbent article, finally coming to fully cover the facing edge portions and to cover a substantial portion of the outer perimeter of the absorbent article as illustrated in FIG. 8.

In making the folding transformation from the configuration shown in FIG. 6, through the configuration shown in FIG. 7, to the configuration shown in FIG. 8, the first cavity (96) is fully opened, second cavity (98) is formed, and the retainer flap (67) is fully turned inside out such that upper surface (86) is disposed outwardly of the absorbent article, and lower surface (88) is disposed inwardly of the second cavity (98). While the first cavity (96) is between the retainer flap (67) and the baffle (64) and is otherwise empty, the second cavity (98) encloses and retains therein a major portion of the outer edge (84) of the absorbent article, along with a corresponding major portion of the body-facing surface, and portions of the absorbent (66) and the baffle (64). In such regard, the second cavity (98) is defined by the combination of the first (90) and second (94) portions of the outer perimeter of the retainer flap (67). As seen in FIG. 8, the mid-section (74) of the absorbent article extends outwardly from the second cavity and defines a portion of the outer surface of the absorbent article as so folded. The first cavity (96) is so defined that, when the second cavity (98) is formed, the absorbent (66), where the retainer flap (67) defines the first cavity (96), remains between the baffle (64) and the cover (62).

While extensibility is not essential to operability of the retainer flap (67), in embodiments where the retainer flap (67) is extensible, such extensibility facilitates the folding over and retention process illustrated in FIGS. 6-8. Where the retainer flap is resiliently extensible, the resilience of the retainer flap material actively retracts about the facing opposing portions of the outer edge of the absorbent article at the second cavity (98) as and after the second cavity (98) is fully formed with the opposing edge portions disposed in the second cavity (98).

The retainer flap (67) can cover any desired portion of the surface area of e.g. the baffle (64) and/or the surface of the absorbent article which is otherwise disposed away from the body-facing surface, so long as the size, placement, and resilience of the retainer flap, and the shape and configuration of the absorbent article, in combination, are operable for performing the fold-over process illustrated in FIGS. 6-8 to thereby retain the facing opposing portions of the outer edge at the second cavity.

Thus, the retainer flap (67) can cover as little as e.g. about 15 percent to about 20 percent of the area of a major surface of the absorbent article, or can cover greater fractions of the area of the absorbent article, for example 40 percent, alternatively up to about 50 percent or greater. Thus, the retainer flap can cover any portion, from about 15 percent to about 50 percent or more, of a major surface of the absorbent article.

As illustrated in FIGS. 7 and 8, the absorbent articles (40) of the invention can carry printed indicia such as a desired graphic or text message, or both on one or more of the inside surfaces of the first cavity (96). The indicia illustrated in part in FIG. 7 is a text message which reads DISPOSE OF PROPERLY. Such message or other indicia is then exposed when the first cavity is opened up in formation of the second cavity as the absorbent article is being prepared for disposal. Such indicia can be, for example, and without limitation, on an inner surface of the retainer flap (67) or on an outer surface, e.g. upper surface (86), of the baffle (64), “inner” representing an orientation toward the absorbent (66) and “outer” representing an orientation away from the absorbent.

FIGS. 9-17 represent a variety of embodiments of labial pads of the invention, and illustrate non-limiting examples of the wide variety of pad shapes which are contemplated for such absorbent articles, as well as the wide range of dispositions and orientations for retainer flaps on such absorbent articles.

As illustrated, for example, in FIG. 13, the facing opposing edge portions, when the used absorbent article is folded with the body-facing side facing inwardly for disposal, need not be coextensive with each other so long as both edge portions are retained in the second cavity (98).

Those skilled in the art will now see that certain modifications can be made to the apparatus and methods herein disclosed with respect to the illustrated embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

Having thus described the invention, what is claimed is:

1. An absorbent article, comprising:
(a) a liquid permeable cover;
(b) a liquid impermeable baffle;
(c) an absorbent disposed between the cover and said baffle; and
(d) a retainer flap extending over said baffle such that said baffle is between said retainer flap and said absorbent,
said absorbent article being configured for disposition primarily within the vestibule of a female wearer,
said absorbent article having a central longitudinal axis, an outer edge defining a first outer perimeter of said absorbent article, a body-facing surface, and a surface opposed to the body-facing surface,
said retainer flap having a second outer perimeter, the second outer perimeter having first and second portions, the first portion being disposed proximate the first outer perimeter of said absorbent article, and being generally attached in said absorbent article at or adjacent the first outer perimeter, the second portion of the second outer perimeter of said retainer flap extending across an outer surface of said baffle whereby said retainer flap covers a portion of said baffle, the second portion of the second outer perimeter of said retainer flap being displaceable from said baffle thereby to define a first cavity between said retainer flap and said baffle.

2. An absorbent article as in claim 1, said absorbent article having a length, said retainer flap being disposed generally on a first side of the central longitudinal axis, and extending along the length of the absorbent article

3. An absorbent article as in claim 1, the second portion of the second outer perimeter of said retainer flap extending generally parallel to the central longitudinal axis.

4. An absorbent article as in claim 1, the second portion of the second outer perimeter of said retainer flap extending in a direction not parallel to the central longitudinal axis.

5. An absorbent article as in claim 1 wherein said retainer flap forms a portion of the surface opposed to the body-facing surface.

6. An absorbent article as in claim 1, said retainer flap comprising at least about 15 percent, and up to about 50 percent, of the surface opposed to the body-facing surface of said absorbent article.

7. An absorbent article as in claim 6, said retainer flap comprising at least about 20 percent of the surface opposed to the body-facing surface of said absorbent article.

8. An absorbent article as in claim 1 wherein, for disposal, said absorbent article is adapted to be folded upon itself such that first and second opposing portions of the outer edge at the first outer perimeter are brought into face-to-face relationship with each other, and wherein said retainer flap is adapted to be folded over the opposing portions of the outer edge to thereby open the first cavity and define a second cavity receiving both of the first and second opposing portions of the outer edge thereinto with said absorbent article so folded, whereby said retainer flap retains the first and second opposing portions of the outer edge in the second cavity, and wherein a mid-section of said absorbent article extends outwardly from the second cavity and defines a portion of an outer surface of said absorbent article as so folded.

9. An absorbent article as in claim 8 wherein said retainer flap is resiliently extensible and wherein resilient forces actively retract the retainer flap about the first and second opposing portions of the outer edge at the second cavity after the second cavity is fully formed with the first and second opposing portions of the outer edge in the second cavity.

10. An absorbent article as in claim 8 wherein a first cavity portion of said absorbent article underlying the said retainer flap at the first cavity is configured such that the absorbent is retained between said baffle and said cover after said second cavity is formed.

11. An absorbent article as in claim 1 wherein the surface opposed to the body-facing surface is defined in part by said retainer flap and in part by said baffle.

12. An absorbent article as in claim 1 wherein the absorbent comprises superabsorbent polymer.

13. An absorbent article as in claim 1 wherein the first portion of the second outer perimeter extends along the first outer perimeter of said absorbent article between first and second ends of the second portion of the second outer perimeter of said retainer flap, whereby said baffle and the first portion of the second outer perimeter of said retainer flap define a closed end of the first cavity.

14. An absorbent article as in claim 1, including printed indicia on a surface which assists in defining, and which is disposed within, the first cavity.

15. An absorbent article, comprising:
(a) a liquid impermeable baffle;
(b) an absorbent; and
(d) a retainer flap extending over said baffle such that said baffle is between said retainer flap and said absorbent,
said absorbent article being configured for disposition primarily within the vestibule of a female wearer,
said absorbent article having a central longitudinal axis, an outer edge defining a first outer perimeter of said absorbent article, a body-facing surface, and a surface opposed to the body-facing surface,
said retainer flap having a second outer perimeter, the second outer perimeter having first and second portions, the first portion being disposed proximate the first outer perimeter of said absorbent article, and being generally attached in said absorbent article at or adjacent the first outer perimeter, the second portion of the outer perimeter of said retainer flap extending across an outer surface of said baffle whereby said retainer flap covers a portion of said baffle, the second portion of the second outer perimeter of said retainer flap being displaceable from said baffle thereby to define a first cavity between said retainer flap and said baffle.

16. An absorbent article as in claim 15, said absorbent article having a length, said retainer flap being disposed generally on a first side of the central longitudinal axis, and extending along the length of the absorbent article

17. An absorbent article as in claim 15, the second portion of the second outer perimeter of said retainer flap extending generally parallel to the central longitudinal axis.

18. An absorbent article as in claim 15, the second portion of the second outer perimeter of said retainer flap extending in a direction not parallel to the central longitudinal axis.

19. An absorbent article as in claim 15 wherein said retainer flap forms a portion of the surface opposed to the body-facing surface.

20. An absorbent article as in claim 15, said retainer flap comprising at least about 15 percent and up to about 50 percent of the surface opposed to the body-facing surface of said absorbent article.

21. An absorbent article as in claim 20, said retainer flap comprising at least about 20 percent of the surface opposed to the body-facing surface of said absorbent article.

22. An absorbent article as in claim 15 wherein, for disposal, said absorbent article is adapted to be folded upon itself such that first and second opposing portions of the outer edge at the first outer perimeter are brought into face-to-face relationship with each other, and wherein said retainer flap is adapted to be folded over the opposing portions of the outer edge to thereby open the first cavity and define a second cavity receiving both of the first and second opposing portions of the outer edge thereinto with said absorbent article so folded, whereby said retainer flap retains the first and second opposing portions of the outer edge in the second cavity, and wherein a mid-section of said absorbent article extends outwardly from the second cavity and defines a portion of an outer surface of said absorbent article as so folded.

23. An absorbent article as in claim 22 wherein said retainer flap is resiliently extensible and wherein resilient forces actively retract the retainer flap about the first and second opposing portions of the outer edge at the second cavity after the second cavity is fully formed with the first and second opposing portions of the outer edge in the second cavity.

24. An absorbent article as in claim 22 wherein a first cavity portion of said absorbent article underlying said retainer flap at the first cavity is configured such that said baffle forms a corresponding portion of the surface opposing the body-facing surface after said second cavity is formed.

25. An absorbent article as in claim 15 wherein the surface opposed to the body-facing surface is defined in part by said retainer flap and in part by said baffle.

26. An absorbent article as in claim 15, wherein the absorbent comprises superabsorbent polymer.

27. An absorbent article, comprising:
(a) a liquid permeable cover;
(b) a liquid impermeable baffle;
(c) an absorbent disposed between the cover and said baffle; and
(d) a retainer flap extending over said baffle such that said baffle is between said retainer flap and said absorbent,
said absorbent article being configured for disposition primarily within the vestibule of a female wearer,
said absorbent article having a central longitudinal axis, an outer edge defining a first outer perimeter of said absorbent article, a body-facing surface, and a surface opposed to the body-facing surface,
said retainer flap having a second outer perimeter, the second outer perimeter having first and second portions, the first portion having first and second separate and distinct sections spaced from each other and disposed proximate the first outer perimeter of said absorbent article at spaced locations on the first outer perimeter, said retainer flap being generally attached in said absorbent article at the first and second sections of the first portion of the second outer perimeter, the second portion of the second outer perimeter of said retainer flap having first and second sections thereof, spaced from each other across a width of said retainer flap and spaced from the first outer perimeter of said absorbent article, and extending across an outer surface of said baffle, thereby to define an open-ended first cavity between said retainer flap and said baffle.

28. An absorbent article as in claim 27, said absorbent article having a length, said retainer flap being disposed generally on a first side of the central longitudinal axis, and extending along the length of the absorbent article

29. An absorbent article as in claim 27, the first and second sections of the second portion of the second outer perimeter of said retainer flap extending generally parallel to the central longitudinal axis.

30. An absorbent article as in claim 27, the first and second sections of the second portion of the second outer perimeter of said retainer flap extending in a direction not parallel to the central longitudinal axis.

31. An absorbent article as in claim 27 wherein said retainer flap forms a portion of the surface opposed to the body-facing surface.

32. An absorbent article as in claim 27, said retainer flap comprising at least about 15 percent, and up to about 40 percent, of the surface opposed to the body-facing surface of said absorbent article.

33. An absorbent article as in claim 27 wherein, for disposal, said absorbent article is adapted to be folded upon itself such that first and second opposing portions of the outer edge at the first outer perimeter are brought into face-to-face relationship with each other, and wherein said retainer flap is adapted to be folded over the opposing portions of the outer edge to thereby open the open-ended first cavity and define an open-ended second cavity receiving both of the first and second opposing portions of the outer edge thereinto with said absorbent article so folded, whereby said retainer flap retains the first and second opposing portions of the outer edge in the open-ended second cavity, and wherein a mid-section of said absorbent article extends outwardly from the open-ended second cavity and defines a portion of an outer surface of said absorbent article as so folded.

34. An absorbent article as in claim 33 wherein said retainer flap is resiliently extensible and wherein resilient forces actively retract the retainer flap about the first and second opposing portions of the outer edge at the second cavity after the second cavity is fully formed with the first and second opposing portions of the outer edge received in the second cavity.

35. An absorbent article as in claim 33 wherein a first cavity portion of said absorbent article underlying the said retainer flap at the first cavity is configured such that the absorbent is retained between said baffle and said cover after said second cavity is formed.

36. An absorbent article as in claim 27 wherein the surface opposed to the body-facing surface is defined in part by said retainer flap and in part by said baffle.

37. An absorbent article as in claim 27 wherein the absorbent comprises superabsorbent polymer.

38. A method of hygienically preparing a labial pad for disposal, the labial pad having a body-facing surface and a surface opposing the body-facing surface, the method comprising:
a) obtain a labial pad ready for disposal, the pad having a cover defining at least a portion of the body-facing surface, a baffle defining a portion of the surface opposing the body-facing surface, an absorbent between the cover and the baffle, and a retainer flap mounted in the labial pad and overlying a portion of the baffle such that the surface opposing the body-facing surface is defined in part by the retainer flap and in part by the baffle, the labial pad having an outer edge defining a first outer perimeter of the labial pad, the retainer flap having a second outer perimeter including a first portion secured in the labial pad at or adjacent the first outer perimeter and a second portion extending across an outer surface of the baffle, the second portion of the second outer perimeter of sais retainer flap being displaceable from the baffle thereby to define a first cavity between the retainer flap and the baffle;
b) folding the labial pad and thereby folding the body-facing surface upon itself such that first and second opposing portions of the outer edge are brought into facing relationship to each other; and
c) folding the retainer flap over both of the folded over first and second opposing portions of the outer edge and thereby opening the first cavity and correspondingly defining a second cavity which receives thereinto both of the first and second facing opposing portions of the outer edge, with the labial pad in the folded condition, whereby the retainer flap assists in retaining the first and second facing opposing portions of the outer edge in the second cavity.

39. A method as in claim 38 wherein a first cavity portion of the absorbent article underlying the retainer flap at the first cavity comprises the baffle forming a corresponding portion of the surface opposing the body-facing surface after the second cavity is formed.

40. A method as in claim 38 wherein the retainer flap is resiliently extensible, the method including resiliently stretching the retainer flap while folding the retainer flap over the folded over first and second opposing portions of the outer edge, and whereby residual resilient forces in the retainer flap actively retract the retainer flap about the first and second opposing portions of the outer edge at the second cavity after the second cavity is fully formed with the first and second opposing portions of the outer edge in the second cavity.
